SEQUENCE LISTING (1 sequence)

SEQ ID NO: 1 - DNA, 1536 bp, synthetic construct
  source 1..1536: mol_type = other DNA; note = Gene encoding the ATP sulfurylase
atgcctgctcctcacggtggtattctacaagacttgattcgtaacaaggcgttaaagaagaatgaattgttatctgaagcgcaatcttcggacattttagtatggaacttgactcctagacaactatgtgatattgaattgattctaaatggtgggttttctcctctgactgggtttttgaacgaaaacgattactcctctgttgttacagattcgagattagcagacggcacattgtggaccatccctattacattagatgttgatgaagcatttgctaaccaaattaaaccagacacaagaattgcccttttccaagatgatgaaattcctattgctatacttactgtccaggatgtttacaagccaaacaaaactatcgaagccgaaaaagtcttcagaggtgacccagaacatccagccattagctatttatttaacgttgccggtgattattacgtcggcggttctttagaagcgattcaattacctcaacattatgactatccaggtttgcgtaagacacctgcccaactaagacttgaattccaatcaagacaatgggaccgtgtcgtagctttccaaactcgtaatccaatgcatagagcccacagggagttgactgtgagagccgccagagaagctaatgctaaggtgctgatccatccagttgttggactaaccaaaccaggtgatatagaccatcacactcgtgttcgtgtctaccaggaaattattaagcgttatcctaatggtattgctttcttatccctgttgccattagcaatgagaatgagtggtgatagagaagccgtatggcatgctattattagaaagaattatggtgcctcccacttcattgttggtagagaccatgcgggcccaggtaagaactccaagggtgttgatttctacggtccatacgatgctcaagaattggtcgaatcctacaagcatgaactggacattgaagttgttccattcagaatggtcacttatttgccagacgaagaccgttatgctccaattgatcaaattgacaccacaaagacgagaaccttgaacatttcaggtacagagttgagacgccgtttaagagttggtggtgagattcctgaatggttctcatatcctgaagtggttaaaatcctaagagaatccaacccaccaagaccaaaacaaggtttttcaattgttttaggtaattcattaaccgtttctcgtgagcaattatccattgctttgttgtcaacattcttgcaattcggtggtggcaggtattacaagatctttgaacacaataataagacagagttactatctttgattcaagatttcattggttctggtagtggactaattattccaaatcaatgggaagatgacaaggactctgttgttggcaagcaaaacgtttacttattagatacctcaagctcagccgatattcagctagagtcagcggatgaacctatttcacatattgtacaaaaagttgtcctatgtctgaaagacaatggcttttttgtattttaa

(19) United States
(12) Patent Application Publication
(10) Pub. No.: US 2026/0286420 A1
(43) Pub. Date: Sep. 24, 2026

(54) REAGENT, KIT, METHOD FOR DETECTING ADP, AND USE THEREOF

(71) Applicant: Neubo Apptech Co., Ltd., Zhejiang (CN)

(72) Inventor: Haiqing Jiang, Zhejiang (CN)

(73) Assignee: Neubo Apptech Co., Ltd., Zhejiang (CN)

(21) Appl. No.: 19/423,289

(22) Filed: Dec. 17, 2025

(30) Foreign Application Priority Data
Mar. 24, 2025 (CN) 202510352914.2

(51) Int. Cl. C12Q 1/48; C12Q 1/34; C12Q 1/66

(52) U.S. Cl. CPC C12Q 1/48; C12Q 1/34; C12Q 1/485; C12Q 1/66

(57) ABSTRACT

A reagent, a kit, a method for detecting ADP, and use thereof are provided. The reagent for detecting ADP includes an ATP sulfurylase, a pyrophosphatase, an enzyme inhibitor, an enzyme that catalyzes the conversion of ADP to ATP, a substrate for the enzyme, a bioluminescent enzyme, and a substrate for the bioluminescent enzyme. The enzyme inhibitor inhibits the activity of ATP sulfurylase and pyrophosphatase.

### CROSS-REFERENCE TO RELATED APPLICATION

This patent application claims the benefit and priority of Chinese Patent Application No. 202510352914.2 filed with the China National Intellectual Property Administration on Mar. 24, 2025, the disclosure of which is incorporated by reference herein in its entirety as part of the present application.

### REFERENCE TO SEQUENCE LISTING

A computer readable XML file entitled “GWP20251002378_seglist”, which was created on Nov. 14, 2025, with a file size of about 3,492 bytes, contains the sequence listing for this application, has been filed with this application, and is hereby incorporated by reference in its entirety.

### TECHNICAL FIELD

The present disclosure belongs to the technical field of ADP detection, and in particular relates to a reagent, a kit, a method for detecting ADP, and use thereof.

### BACKGROUND

Phosphotransferases, also known as phosphorylases, are a class of enzymes that use energy molecules, such as adenosine triphosphate (ATP), to add a phosphate group to a corresponding substrate molecule. ATP hydrolases are enzymes that break down ATP to generate ADP and a phosphate group (Pi), and release energy.

Kinases are a type of phosphotransferases and have important physiological functions. They mainly participate in phosphotransfer reactions in cells, which are key steps in cell signaling and metabolism. Through the catalytic action of kinases, cells are able to modulate the internal metabolic processes, ensuring the smooth progress of various biochemical reactions. Kinases primarily include protein kinases, lipid kinases, and sugar kinases based on the categories of substrates they act on. They are highly specific, meaning they can only catalyze specific chemical reactions. They have a strong affinity towards reactants and can bind tightly to them, thereby accelerating reactions. Currently, studies of kinases have become one of the hot topics in the field of medical research. Many drugs are designed to target specific kinases in order to modulate their activity for therapeutic purposes. Therefore, highly sensitive and specific detection of kinase activity is particularly important for studying kinase functions.

In recent decades, methods have been developed for detecting kinase activity including fluorescence resonance energy transfer (FRET) assays, fluorescence polarization (FP) assays, and radioactivity-based assays such as scintillation proximity assays (SPA). FRET-based assays have many drawbacks, including a high rate of false positives, fluorescent interference from measured compounds, a narrow dynamic range, and performance issues associated with the antibody employed in the assay, e.g., specificity, etc. FP assays are susceptible to significant interference from phosphorylated proteins and other reaction components, such as lipids and detergents, which affects the reproducibility of detections. Similarly, the use of SPA assays is limited by factors such as the use of isotopes, the use of specific substrates, and antibody performance.

To develop kinase detection methods with high specificity, sensitivity, and broad application, attempts have been made to use bioluminescence method to measure the adenosine diphosphate (ADP) level in kinase or ATP hydrolase reactions, thereby more accurately reflecting the activity of relevant enzymes. For example, Shultz et al. published an article in 1993 (Bioluminometric Assay of ADP and ATP at High ATP/ADP Ratios: Assay of ADP after Removal of ATP). However, there are two shortcomings in the Shultz's method: 1) The efficiency of ATP sulfurylase (ATPS) in removing ATP during the ATP removal step needs to be further improved, because even a residual 0.5% of ATP will interfere with the background of subsequent ADP detection and reduce the signal-to-noise ratio of the experiment, particularly in kinase reaction systems at low ATP concentrations; 2) Inactivating ATP sulfurylase in boiling water after ATP removal complicates the experimental operation, and inactivation with boiling water does not facilitate the application to microplate detections and high-throughput detection experiments.

### SUMMARY

In this regard, an object of the present disclosure is to provide a reagent for detecting ADP. The reagent has high accuracy and sensitivity in the detection of ADP and stable fluorescence signals. The reagent may be used to detect the enzyme activity of a phosphotransferase and/or an ATP hydrolase.

To achieve the foregoing object, the present disclosure provides the following technical solutions.

Provided is a reagent for detecting ADP, including an ATP sulfurylase, a pyrophosphatase, an enzyme inhibitor, an enzyme that catalyzes the conversion of ADP to ATP, a substrate for the enzyme, a bioluminescent enzyme, and a substrate for the bioluminescent enzyme, where the enzyme inhibitor inhibits activities of the ATP sulfurylase and the pyrophosphatase.

In some embodiments, the nucleotide sequence of a gene encoding the ATP sulfurylase is set forth in SEQ ID NO: 1.

In some embodiments, the enzyme that catalyzes the conversion of ADP to ATP is any one of a myokinase, a creatine kinase, or a pyruvate kinase, and the substrate of the enzyme is a phosphate donor that is capable of being used by the enzyme.

In some embodiments, the enzyme inhibitor includes EDTA and EGTA.

Another object of the present disclosure is to provide a kit for detecting ADP, comprising the above reagent and further an ADP/ATP mixture standard.

Another object of the present disclosure is to provide a method for detecting ADP, which uses the reagent or the kit for detection, comprising steps as follows: removing ATP from a sample using an ATP sulfurylase and a pyrophosphatase; converting ADP in the sample into ATP using an enzyme that catalyzes the conversion of ADP to ATP; and detecting ATP in the sample using a bioluminescence reaction.

In some embodiments, after removing ATP from the sample, the activity of ATP sulfurylase and pyrophosphatase is inhibited using the enzyme inhibitor.

Another object of the present disclosure is to provide use of the reagent, the kit, or the method for detecting the activity of an enzyme, where the enzyme includes a phosphotransferase and an ATP hydrolase.

Another object of the present disclosure is to provide use of the reagent, the kit, or the method for detecting and/or screening for an enzyme activity modulator, where the enzyme includes a phosphotransferase and an ATP hydrolase.

In some embodiments, the modulator includes an enzyme inhibitor and an enzyme activator.

Compared with the prior art, embodiments of the present disclosure has the following beneficial effects.

The present disclosure provides a reagent for detecting ADP, useful for the qualitative or quantitative detection of ADP in a sample. Using the ATP-removal reagent of the present disclosure, the residual ATP in the kinase reaction can be efficiently depleted to less than 0.01% of the initial amount. Moreover, the chemical inactivation method adopted not only effectively quenches the activities of ATP sulfurylase and pyrophosphatase in the reaction, but also exerts no interference with the subsequent steps. The luminescent signal generated in the detection of ADP using the reagent of the present disclosure is stable and is applicable to long-term detection. ADP in a sample to be detected can be generated from the consumption of the substrate ATP by a phosphotransferase and/or an ATP hydrolase. The enzyme activity is positively correlated with the yield of ADP. In the present disclosure, the enzyme activity of a phosphotransferase and/or an ATP hydrolase is determined by detecting the content of ADP. Therefore, embodiments of the present disclosure can be used to detect the enzyme activity of a phosphotransferase and/or an ATP hydrolase, and has the advantages of high sensitivity, excellent linearity, and stable fluorescence signal.

### BRIEF DESCRIPTION OF THE DRAWINGS

FIG. 1 shows results of the sodium dodecyl sulfate-polyacrylamide gel electrophoresis (SDS-PAGE) of the purified ATP sulfurylase.

FIG. 2 shows the standard curves of the ADP/ATP mixture in the 1 mM series, where the left panel shows the readings after 30 minutes of incubation, and the right panel shows the stability and linearity of the readings within 3 hours.

FIG. 3 shows the standard curves of the ADP/ATP mixture in the 100 μM series, where the left panel shows the readings after 30 minutes of incubation, and the right panel shows the stability and linearity of the readings within 3 hours.

FIG. 4 shows the standard curves of the ADP/ATP mixture in the 10 μM series, where the left panel shows the readings after 30 minutes of incubation, and the right panel shows the stability and linearity of the readings within 3 hours.

FIG. 5 shows the standard curves of the ADP/ATP mixture in the 1 μM series, where the left panel shows the readings after 30 minutes of incubation, and the right panel shows the stability and linearity of the readings within 3 hours.

FIG. 6 shows the dose curve and EC₅₀ of H8-9 under the conditions of 10 μM ATP, where the left panel shows the readings after 30 minutes of incubation, and the right panel shows stability of the readings within 3 hours.

FIG. 7 shows the dose curve and EC₅₀ of H8-9 under the laboratory conditions of 100 μM ATP, where the left panel shows the readings after 30 minutes of incubation, and the right panel shows stability of the readings within 3 hours.

FIG. 8 shows the dose curve and EC₅₀ of cAMP-dependent protein kinase inhibitor (PKI) under the laboratory conditions of 10 μM ATP, where the left panel shows the readings after 30 minutes of incubation, and the right panel shows stability of the readings within 3 hours.

FIG. 9 shows the dose curve and EC₅₀ of PKI under the laboratory conditions of 100 μM ATP, where the left panel shows the readings after 30 minutes of incubation, and the right panel shows stability of the readings within 3 hours.

FIG. 10 shows the dose curve and EC₅₀ of STSP under the laboratory conditions of 1 μM and 10 μM ATP, where the left panel shows the ATP concentration of 1 μM, and the right panel shows the ATP concentration of 10 μM.

### DETAILED DESCRIPTION OF THE EMBODIMENTS

The present disclosure provides a reagent for detecting ADP comprises an ATP sulfurylase, a pyrophosphatase, an enzyme inhibitor, an enzyme that catalyzes the conversion of ADP to ATP, a substrate for the enzyme, a bioluminescent enzyme, and a substrate for the bioluminescent enzyme. The enzyme inhibitor inhibits the activity of ATP sulfurylase and pyrophosphatase.

In the present disclosure, ATP sulfurylase includes ATP sulfurylase produced from various expression systems and natural ATP sulfurylase from various species. The ATP sulfurylase produced from various expression systems includes ATP sulfurylase produced from any of a bacterial expression system, a yeast expression system, an insect cell expression system, a mammalian cell expression system, and a cell-free in vitro expression system; the natural ATP sulfurylase includes any of bacterial ATP sulfurylase, fungal ATP sulfurylase, mammalian ATP sulfurylase, and plant ATP sulfurylase. In the present disclosure, the nucleotide sequence of a gene encoding ATP sulfurylase is preferably set forth in SEQ ID NO: 1.

ATGCCTGCTCCTCACGGTGGTATTCTACAAGACTTGATTCGTAACAAG
GCGTTAAAGAAGAATGAATTGTTATCTGAAGCGCAATCTTCGGACATT
TTAGTATGGAACTTGACTCCTAGACAACTATGTGATATTGAATTGATT
CTAAATGGTGGGTTTTCTCCTCTGACTGGGTTTTTGAACGAAAACGAT
TACTCCTCTGTTGTTACAGATTCGAGATTAGCAGACGGCACATTGTGG
ACCATCCCTATTACATTAGATGTTGATGAAGCATTTGCTAACCAAATT
AAACCAGACACAAGAATTGCCCTTTTCCAAGATGATGAAATTCCTATT
GCTATACTTACTGTCCAGGATGTTTACAAGCCAAACAAAACTATCGAA
GCCGAAAAAGTCTTCAGAGGTGACCCAGAACATCCAGCCATTAGCTAT
TTATTTAACGTTGCCGGTGATTATTACGTCGGCGGTTCTTTAGAAGCG
ATTCAATTACCTCAACATTATGACTATCCAGGTTTGCGTAAGACACCT
GCCCAACTAAGACTTGAATTCCAATCAAGACAATGGGACCGTGTCGTA
GCTTTCCAAACTCGTAATCCAATGCATAGAGCCCACAGGGAGTTGACT
GTGAGAGCCGCCAGAGAAGCTAATGCTAAGGTGCTGATCCATCCAGTT
GTTGGACTAACCAAACCAGGTGATATAGACCATCACACTCGTGTTCGT
GTCTACCAGGAAATTATTAAGCGTTATCCTAATGGTATTGCTTTCTTA
TCCCTGTTGCCATTAGCAATGAGAATGAGTGGTGATAGAGAAGCCGTA
TGGCATGCTATTATTAGAAAGAATTATGGTGCCTCCCACTTCATTGTT
GGTAGAGACCATGCGGGCCCAGGTAAGAACTCCAAGGGTGTTGATTTC
TACGGTCCATACGATGCTCAAGAATTGGTCGAATCCTACAAGCATGAA
CTGGACATTGAAGTTGTTCCATTCAGAATGGTCACTTATTTGCCAGAC
GAAGACCGTTATGCTCCAATTGATCAAATTGACACCACAAAGACGAGA
ACCTTGAACATTTCAGGTACAGAGTTGAGACGCCGTTTAAGAGTTGGT
GGTGAGATTCCTGAATGGTTCTCATATCCTGAAGTGGTTAAAATCCTA
AGAGAATCCAACCCACCAAGACCAAAACAAGGTTTTTCAATTGTTTTA
GGTAATTCATTAACCGTTTCTCGTGAGCAATTATCCATTGCTTTGTTG
TCAACATTCTTGCAATTCGGTGGTGGCAGGTATTACAAGATCTTTGAA
CACAATAATAAGACAGAGTTACTATCTTTGATTCAAGATTTCATTGGT
TCTGGTAGTGGACTAATTATTCCAAATCAATGGGAAGATGACAAGGAC
TCTGTTGTTGGCAAGCAAAACGTTTACTTATTAGATACCTCAAGCTCA
GCCGATATTCAGCTAGAGTCAGCGGATGAACCTATTTCACATATTGTA
CAAAAAGTTGTCCTATGTCTGAAAGACAATGGCTTTTTTGTATTTTAA.

In the present disclosure, the enzyme that catalyzes the conversion of ADP to ATP is any one of a myokinase, a creatine kinase, or a pyruvate kinase, and the substrate for the enzyme is a phosphate donor that can be used by the enzyme. In some embodiments, the enzyme is a pyruvate kinase, and the substrate for the enzyme is potassium phosphoenolpyruvate.

In the present disclosure, the enzyme inhibitor includes EDTA (ethylenediaminetetraacetic acid) and EGTA (ethylene glycol diethyl ether diamine tetraacetic acid). The present disclosure utilizes an enzyme inhibitor to inactivate ATP sulfurylase and pyrophosphatase, making it applicable to microplate detections and high-throughput screening.

In the present disclosure, the bioluminescent enzyme is preferably a luciferase, and the substrate for the bioluminescent enzyme is preferably D-luciferin or a D-luciferin salt.

The present disclosure further provides a kit for detecting ADP, including the above reagent; preferably, the kit further includes an ADP/ATP mixture standard.

The present disclosure further provides a method for detecting ADP, which uses the reagent or the kit for detection, including steps as follows: removing ATP from a sample using an ATP sulfurylase and a pyrophosphatase; converting ADP in the sample into ATP using an enzyme that catalyzes the conversion of ADP to ATP; and detecting ATP in the sample using a bioluminescence reaction.

In the present disclosure, when ATP is present in a sample to be tested, it will affect the accuracy of ADP detection. Therefore, it is necessary to remove ATP from the sample. The present disclosure utilizes ATP sulfurylase to convert ATP in the sample into adenosine monophosphate (AMP) and pyrophosphoric acid (PPi) in the presence of sodium molybdate (Na₂MoO₄). Pyrophosphatase further catalyzes PPi to phosphate (Pi), driving the reaction to further convert ATP into AMP, thereby reducing the remaining ATP in the sample to 1%, 0.1%, 0.01%, 0.001%, 0.0001% of the initial content or below, or eliminating it. When ATP is reduced to a minimum amount or eliminated, ADP is converted into ATP using an enzyme capable of converting ADP into ATP, and the converted ATP is then detected using a bioluminescent enzyme and a substrate for the bioluminescent enzyme.

In the present disclosure, preferably upon removal of ATP from the sample with ATP sulfurylase and pyrophosphatase, the enzyme inhibitor is used to inhibit the activity of the ATP sulfurylase and the pyrophosphatase. The method provided by the present disclosure is applicable to microplate detections and high throughput screening.

The method provided by the present disclosure may significantly improve the linear relationship between a compound concentration and signal intensity during detection, and also provide a long half-life for the detection signal. The detection method provided by the present disclosure is non-radioactive, highly sensitive, and convenient, making it applicable to microplate-based high-throughput detections. The luminescent signal generated by the present disclosure is stable and is applicable to long-term detection. The method provided by the present disclosure can be used to detect a wide range of ADP or ATP concentrations. In the present disclosure, ADP or ATP can be detected at concentrations as low as 1 picoM or below, and as high as 10 mM or above.

The present disclosure further provides use of the reagent, the kit, or the method for detecting the activity of an enzyme, where the enzyme includes a phosphotransferase and an ATP hydrolase.

In the present disclosure, when used to detect the activity of a phosphotransferase or an ATP hydrolase, the phosphotransferase or ATP hydrolase uses ATP as a substrate and converts ATP to ADP, and after the removal of the remaining ATP in the reaction, the enzyme activity is determined by detecting the ADP amount.

In the present disclosure, when used to detect the activity of an enzyme, ATP is mixed with the phosphotransferase or ATP hydrolase to form a first mixture including ADP and remaining ATP; the first mixture is mixed with an ATP sulfurylase and a pyrophosphatase to form a second mixture including AMP, Pi, the ATP sulfurylase, the pyrophosphatase, and ADP; the second mixture is mixed with an enzyme inhibitor, an enzyme that catalyzes the conversion of ADP to ATP, a substrate for the enzyme that catalyzes the conversion of ADP to ATP, a bioluminescent enzyme, and a substrate for the bioluminescent enzyme to form a third mixture, and the luminescence intensity of the third mixture is detected. In the present disclosure, the bioluminescent enzyme and the substrate for the bioluminescent enzyme can be added simultaneously with the enzyme inhibitor, the enzyme that catalyzes the conversion of ADP to ATP, and the substrate for the enzyme that catalyzes the conversion of ADP to ATP; alternatively, the bioluminescent enzyme and the substrate for the bioluminescent enzyme can be added after the addition of the enzyme inhibitor, the enzyme that catalyzes the conversion of ADP to ATP, and the substrate for enzyme that catalyzes the conversion of ADP to ATP.

The present disclosure further provides use of the reagent, the kit, or the method for detecting and/or screening for an enzyme activity modulator, where the enzyme includes a phosphotransferase and an ATP hydrolase, and the modulator includes an enzyme inhibitor and an enzyme activator.

In the present disclosure, when used to detect and/or screen for an enzyme activity modulator, ATP is mixed with a phosphotransferase or an ATP hydrolase, or a modulator of the phosphotransferase or ATP hydrolase to form a first mixture including ADP and remaining ATP; the first mixture is mixed with an ATP sulfurylase and a pyrophosphatase to form a second mixture including AMP, Pi, the ATP sulfurylase, the pyrophosphatase, and ADP; the second mixture is mixed with an enzyme inhibitor, an enzyme that catalyzes the conversion of ADP to ATP, a substrate for the enzyme that catalyzes the conversion of ADP to ATP, a bioluminescent enzyme, and a substrate for the bioluminescent enzyme to form a third mixture, and the luminescence intensity of the third mixture is detected. In the present disclosure, the bioluminescent enzyme and the substrate for the bioluminescent enzyme can be added simultaneously with the enzyme inhibitor, the enzyme that catalyzes the conversion of ADP to ATP, and the substrate for the enzyme that catalyzes the conversion of ADP to ATP; alternatively, the bioluminescent enzyme and the substrate for the bioluminescent enzyme can be added after the addition of the enzyme inhibitor, the enzyme that catalyzes the conversion of ADP to ATP, and the substrate for enzyme that catalyzes the conversion of ADP to ATP.

Detailed information about the reagents used in specific examples of the present disclosure is shown in Table 1.

TABLE 1
Reagent Information

| | | | Catalog |
|---|---|---|---|
| No. | Product Name | Manufacturer | No. |
| 1 | LB broth | Merck | 1.10285 |
| 2 | Ni-NTA His Bind Resin | Merck | 70666 |
| 3 | ATP | Merck | A1852 |
| 4 | ADP | Merck | A2754 |
| 5 | Pyrophosphatase | Merck | I5907 |
| 6 | Tris-HCl, pH 7.4 | Shanghai Yuanye | R21406 |
| 7 | Magnesium chloride | Merck | 930989 |
| 8 | Sodium molybdate | Merck | M1003 |
| 9 | Ethylenediaminetetraacetic acid | Merck | 324503 |
| 10 | Ethylene glycol diethyl ether | Merck | 0377 |
| | diamine tetraacetic acid | | |
| 11 | Magnesium sulfate | Merck | 203726 |
| 12 | Potassium chloride | Merck | P3911 |
| 13 | D-luciferin | Merck | L9504 |
| 14 | Phosphoenolpyruvate | Merck | 860077 |
| | monopotassium salt | | |
| 15 | Luciferase | Ningbo Youbo | X2102 |
| 16 | Pyruvate kinase | Shanghai Yuanye | S10172 |
| 17 | Bovine serum albumin (BSA) | Merck | A1933 |
| 18 | Protein kinase A | Merck | P5511 |
| 19 | H8-9 | Merck | H8901 |
| 20 | PKI | Merck | SCP0064 |
| 21 | Staurosporine (STSP) | Merck | 569397 |
| 22 | ADP-Glo Kinase Assay | Promega | V9101 |

The technical solutions provided by the present disclosure will be explained in detail with reference to the following examples. However, these examples shall not be construed as limiting the protection scope of the present disclosure.

### Example 1

Provided is a reagent for detecting ADP including an ATP sulfurylase, a pyrophosphatase, EDTA, EGTA, a pyruvate kinase, a phosphoenolpyruvate monopotassium salt, a luciferase, and D-luciferin.

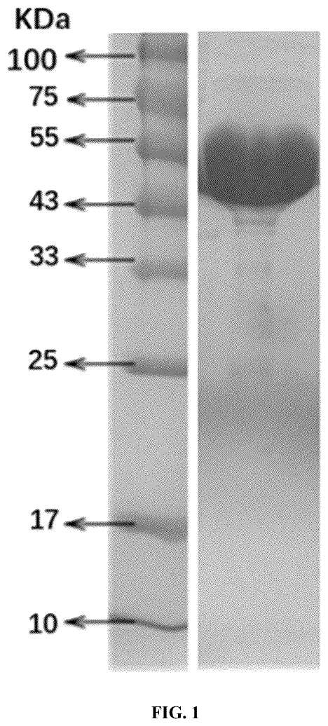

ATP sulfurylase (ATPS) is an enzyme encoded by the ME3 gene of Saccharomyces cerevisiae. The nucleotide sequence of the gene is set forth in SEQ ID NO: 1. This gene and the E. coli expression vector pET 15b were double-digested with NdeI and BamH1, respectively, and then ligated to obtain a recombinant vector. The recombinant vector was transformed into competent cells (BL21, purchased from Thermo Fisher Scientific). A single colony including the recombinant vector was inoculated onto a Luria-Bertani (LB) agar medium. The next day, a colony was picked and inoculated into 2 mL of a LB liquid medium. When the OD₆₀₀ value of the medium reached 0.5, 2 mL of the bacterial solution was inoculated into 1 μL of the LB liquid medium. When the OD₆₀₀ value of the medium reached 2, IPTG (isopropyl-β-D-thiogalactopyranoside) was added to a final concentration of 0.5 mM. Induction was performed for 2 hours. The medium was collected and centrifuged in a centrifuge at 6000 rpm for 10 minutes. The supernatant was removed to collect the bacteria. Then, 50 mL of Tris-HCl buffer with concentration of 50 mM (pH 7.4) was added to the bacteria and sonicated in an sonicator for 2 minutes. After centrifugation, the supernatant was collected and passed through a Ni-NTA column. Elution was performed using a Tris-HCl solution including 200 mM imidazole, and an eluted sample was collected. The electrophoresis of the purified protein is shown in FIG. 1.

### Example 2

The ATP-removal effect of the reagent in Example 1 was verified.

ATP sulfurylase was prepared in the following buffer: 50 mM Tris-HCl buffer (pH 7.4)+10 mM MgCl₂+20 mM Na₂MoO₄, where the concentration of each component is the final concentration in the resultant solution.

Reagent for ADP detection: 50 mM Tris-HCl buffer (pH 7.4)+20 mM MgSO₄+50 mM KCl+2 mM EDTA+2 mM EGTA+0.1 mM D-luciferin+0.1 mM PEP-K (phosphoenolpyruvate monopotassium salt)+50 μg/mL luciferase+20 U/mL a pyruvate kinase, where the concentration of each component is the final concentration in the resultant solution.

The bioluminescence detection instrument was an EnVision (Perkin Elmer).

Next, 25 μL of ATP (100 M) was added to each well of a 96-well white opaque assay plate, followed by the addition of 25 μL of pyrophosphatase (PPA: 10 U/mL) and ATP sulfurylase (ATPS) at concentrations of 2 μg/mL, 1 μg/mL, and 0.5 μg/mL.

After incubation at room temperature (22° C.-25° C.) for 40 minutes, 50 μL of the reagent for ADP detection was added and mixed uniformly, and the fluorescence values were read after incubation was performed at room temperature for 10 minutes.

After 1 μM ATP+0.1 mM D-luciferin+50 μg/mL luciferase+50 mM Tris-HCl buffer (pH 7.4)+20 mM MgSO₄ were incubated at room temperature for 10 minutes, the fluorescence value read was 439,640.

After 0.1 μM ATP+0.1 mM D-luciferin+50 μg/mL luciferase+50 mM Tris-HCl buffer (pH 7.4)+20 mM MgSO₄ were incubated at room temperature for 10 minutes, the fluorescence value read was 47,720.

The remaining ATP after the action of ATPS and PPA was determined by comparing with the fluorescence values under conditions of ATP at 1 μM and 0.1 μM (see Table 2). It can be seen that the remaining ATP of 100 μM ATP under the action of ATPS (1 μg/mL) and pyrophosphatase was about 0.006%.

TABLE 2
Remaining ATP of 100 μM ATP
under the action of ATPS and PPA

| ATPS (μg/mL) | Fluorescence value (RLU) | Remaining ATP (%) |
|---|---|---|
| 2 | 2.24E+03 | 0.0047 |
| 1 | 2.88E+03 | 0.0060 |
| 0.5 | 3.44E+03 | 0.0072 |

### Example 3

### ADP/ATP Mixture Standard Curve Experiment.

The detection was performed using the reagent of Example 1.

Reagent for ATP removal: 50 mM Tris-HCl buffer (pH 7.4)+10 mM MgCl₂+20 mM Na₂MoO₄+1 μg/mL ATPS+10 U/mL PPA, where the concentration of each component is the final concentration in the reagent.

Reagent for ADP detection: 50 mM Tris-HCl buffer (pH 7.4)+20 mM MgSO₄+50 mM KCl+2 mM EDTA+2 mM EGTA+0.1 mM D-luciferin+0.1 mM PEP-K (phosphoenolpyruvate monopotassium salt)+50 μg/mL luciferase+20 U/mL a pyruvate kinase, where the concentration of each component is the final concentration in the reagent.

Universal kinase reaction buffer: 50 mM Tris buffer (pH 7.4)+20 mM MgCl₂+0.1 mg/mL BSA, where the concentration of each component is the final concentration in the buffer.

The bioluminescence detection instrument was an EnVision (Perkin Elmer).

The ADP/ATP mixture standard concentration detection simulated the amounts of remaining ATP and ADP produced after the kinase reactions. The ADP-to-ATP ratios are shown in Table 3.

TABLE 3
ADP-to-ATP ratio

| | | | | | | | | | | | | |
|---|---|---|---|---|---|---|---|---|---|---|---|---|
| % ADP | 100 | 80 | 60 | 40 | 20 | 10 | 5 | 4 | 3 | 2 | 1 | 0 |
| % ATP | 0 | 20 | 40 | 60 | 80 | 90 | 95 | 96 | 97 | 98 | 99 | 100 |

The concentrations of a 1 mM ADP/ATP mixture in a standard curve were prepared by:
diluting both ADP and ATP with the universal kinase reaction buffer to a concentration of 1 mM, and mixing according to the volumes listed in Table 4 to afford a 1 mM ADP/ATP mixture standard concentration series.

TABLE 4
ADP-to-ATP volume ratio

| | | | | | | | | | | | | |
|---|---|---|---|---|---|---|---|---|---|---|---|---|
| 1 mM | 100 | 80 | 60 | 40 | 20 | 10 | 5 | 4 | 3 | 2 | 1 | 0 |
| ADP (μL) | | | | | | | | | | | | |
| 1 mM | 0 | 20 | 40 | 60 | 80 | 90 | 95 | 96 | 97 | 98 | 99 | 100 |
| ATP (μL) | | | | | | | | | | | | |

The 1 mM ADP/ATP mixture standard concentration series was diluted by 10-fold with the universal kinase reaction buffer to afford a 100 μM ADP/ATP mixture standard concentration series.

The 100 μM ADP/ATP mixture standard concentration series was diluted by 10-fold with the universal kinase reaction buffer to afford a 10 μM ADP/ATP mixture standard concentration series.

The 10 μM ADP/ATP mixture standard concentration series was diluted by 10-fold with the universal kinase reaction buffer to afford a 1 μM ADP/ATP mixture standard concentration series.

### Steps of Test:

25 μL of the above-prepared 1 mM/100 μM/10 μM/1 μM ADP/ATP mixture standard concentration series were added to each well of a 96-well white opaque assay plate, respectively, followed by the addition of 25 μL of the reagent for ATP removal. After incubation at room temperature (22° C.-25° C.) for 40 minutes, 50 μL of the reagent for ADP detection was added and mixed uniformly, and the fluorescence values were read after incubation was performed at room temperature for 30 minutes, 1 hour, 2 hours, 3 hours, 16 hours, and 23 hours, respectively.

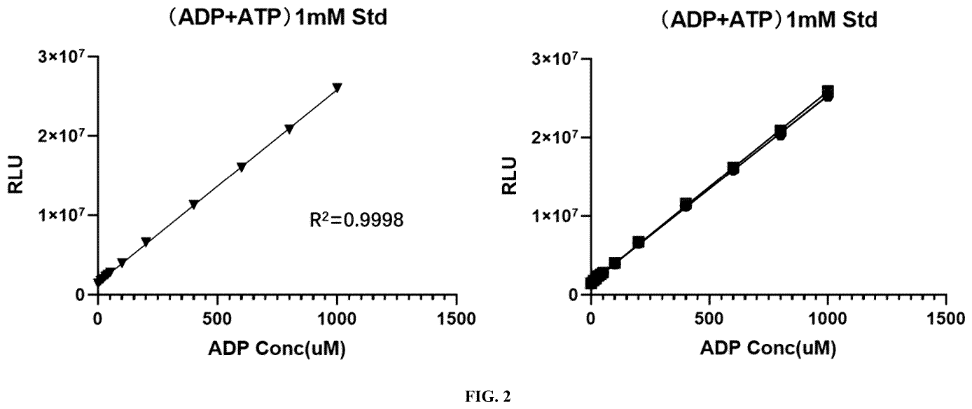

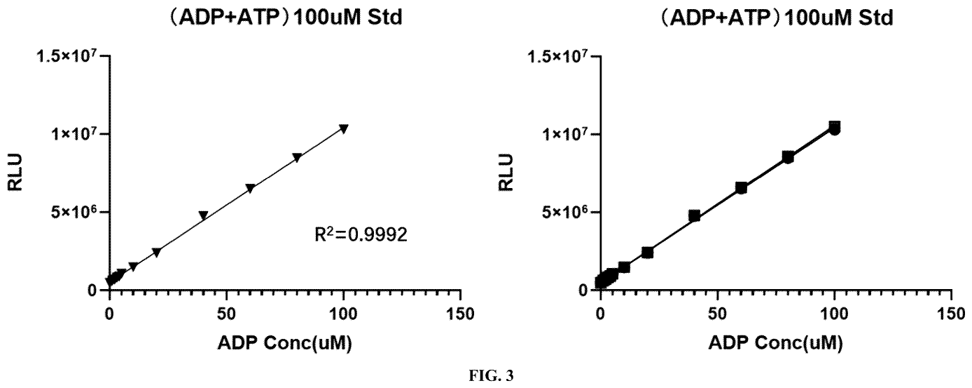

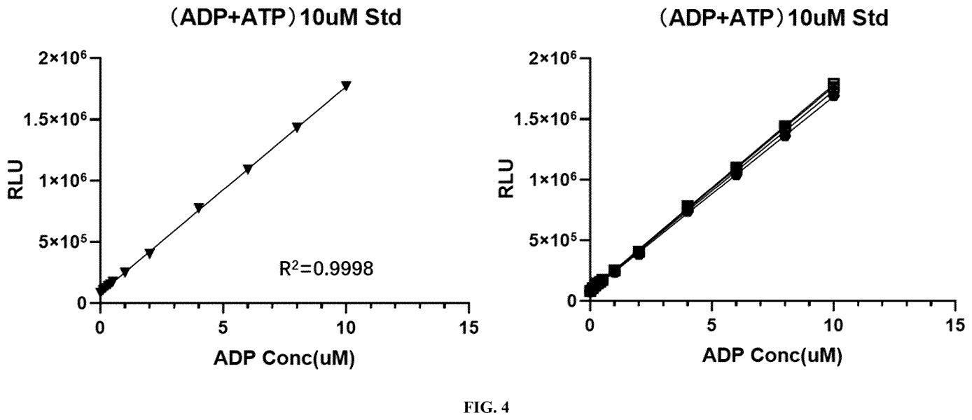

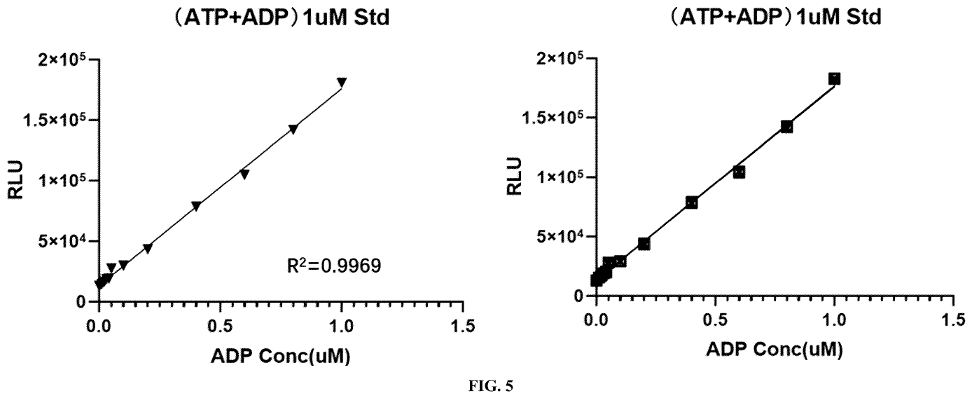

FIG. 2 shows the standard curves of the 1 mM ADP/ATP mixture standard concentration series; FIG. 3 shows the standard curves of the 100 μM ADP/ATP mixture standard concentration series; FIG. 4 shows the standard curves of the 10 μM ADP/ATP mixture standard concentration series; and FIG. 5 shows the standard curves of the 1 μM ADP/ATP mixture standard concentration series. As shown in FIGS. 2-5, the ADP concentrations and fluorescence values of the 1 mM/100 μM/10 μM/1 μM ADP/ATP mixture standard concentration series all showed a linear relationship after fit by GraphPad Prism 8, with R²>0.99. Further, the readings of each concentration series at 30 minutes, 1 hour, 2 hours, and 3 hours almost overlap with the fitted standard curves, indicating that the fluorescence intensity remained unchanged within the 3-hour detection period compared to the fluorescence intensity at the 30-minute incubation.

In addition, the present disclosure showed that when incubated for 23 hours, the reading of a sample with a 100% ADP ratio in the 1 mM ADP/ATP mixture standard concentration series was 1.9×10⁷; and when incubated for 30 minutes, the reading was 2.5×10⁷. The reading of a sample with a 100% ADP ratio in the 100 μM ADP/ATP mixture standard concentration series was 8.2×10⁶; and when incubated for 30 minutes, the reading was 1.1×10⁷. The reading of a sample with a 100% ADP ratio in the 10 μM ADP/ATP mixture standard concentration series was 1.4×10⁶; and when incubated for 30 minutes, the reading was 1.8×10⁶. The reading of a sample with a 100% ADP ratio in the 1 μM ADP/ATP mixture standard concentration series was 1.4×10⁵, and when incubated for 30 minutes, the reading was 1.8×10⁵.

It can be seen that the readings after 16-23 hours are still greater than 70% of the fluorescence intensity at 30-minute incubation, and R²>0.99, indicating that the present disclosure improves the linear relationship between the ADP concentration and signal intensity, and the luminescent signal is stable and is suitable for long-term detection.

### Example 4

The EC₅₀ values of PKA (protein kinase A)-specific inhibitors H8-9 and PKI against PKA were detected.

The detection was performed using the reagent of Example 1.

Reagent for ATP removal: 50 mM Tris-HCl buffer (pH 7.4)+10 mM MgCl₂+20 mM Na₂MoO₄+1 μg/mL ATPS+10 U/mL PPA, where the concentration of each component is the final concentration in the reagent.

Reagent for ADP detection: 50 mM Tris-HCl buffer (pH 7.4)+20 mM MgSO₄+50 mM KCl+2 mM EDTA+2 mM EGTA+0.1 mM D-luciferin+0.1 mM PEP-K+50 μg/mL luciferase+20 U/mL a pyruvate kinase, where the concentration of each component is the final concentration in the reagent.

Universal kinase reaction buffer: 50 mM Tris buffer (pH 7.4)+20 mM MgCl₂+0.1 mg/mL BSA, where the concentration of each component is the final concentration in the buffer.

The bioluminescence detection instrument was an EnVision (Perkin Elmer).

### 1. EC₅₀ of H8-9 Against PKA.

H8-9 was serially diluted by 3-fold using universal kinase reaction buffer, to a maximum concentration of 30 μM and a minimum concentration of 1 nM.

ATP concentration of 10 μM:

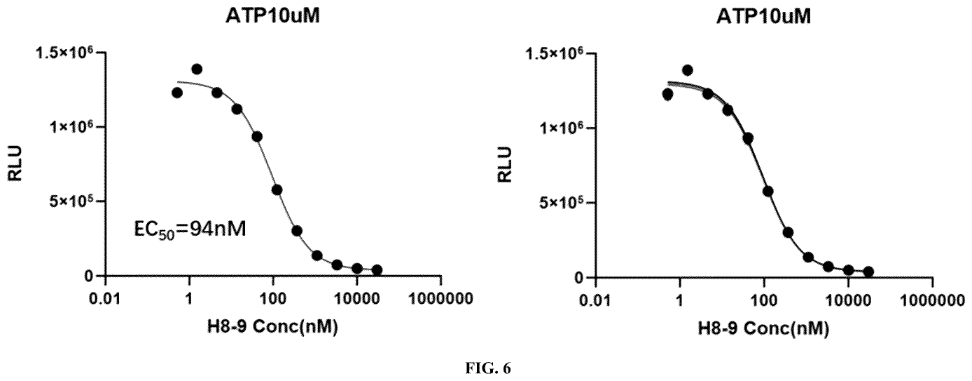

25 μL of the universal kinase reaction buffer including 80 U PKA and a varied concentration of H8-9 was added to each well of a 96-well white opaque assay plate, and 10 μM ATP was added. The reaction was performed at room temperature (22° C.-25° C.) for 10 minutes. After the reaction was finished, 25 μL of the reagent for ATP removal was added. After incubation at room temperature (22° C.-25° C.) for 40 minutes, 50 μL of the reagent for ADP detection was added and mixed uniformly, and the fluorescence values were read after incubation was performed at room temperature for 30 minutes, 1 hour, 2 hours, 3 hours, and 21 hours, respectively. The ADP concentrations and fluorescence values were fitted using GraphPad Prism 8 before the calculation of EC₅₀. The H8-9 dose curve is shown in FIG. 6. Furthermore, the readings for each concentration series at 30 minutes, 1 hour, 2 hour, and 3 hours almost overlap with the dose curve, indicating high stability in fluorescence intensity.

The fluorescence signal of the present disclosure is stable. At an H8-9 concentration of 3 nM, the reading after 21 hours of incubation was 7.5×10⁵, still greater than 50% of the readings at 30 minutes of incubation (1.4×10⁶), and the EC₅₀ remained unchanged.

ATP concentration of 100 μM:

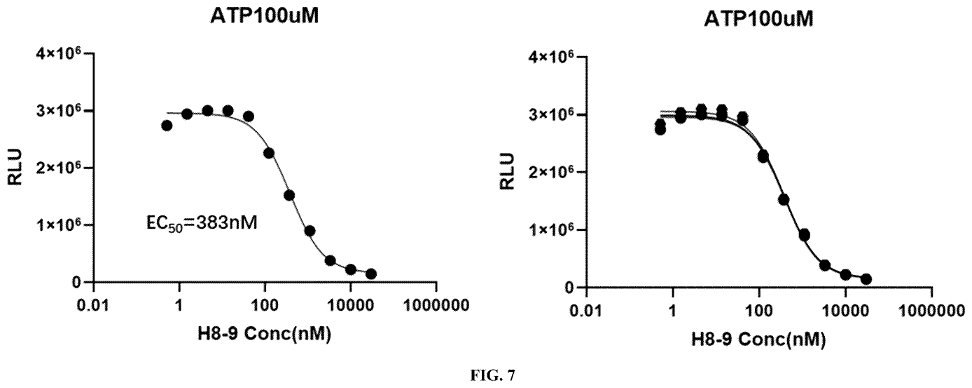

25 μL of the universal kinase reaction buffer including 80 U PKA and a varied concentration of H8-9 was added to each well of a 96-well white opaque assay plate, and 100 μM ATP was added. The reaction was performed at room temperature (22° C.-25° C.) for 30 minutes. After the reaction was finished, 25 μL of the reagent for ATP removal was added. After incubation at room temperature (22° C.-25° C.) for 40 minutes, 50 μL of the reagent for ADP detection was added and mixed uniformly, and the fluorescence values were read after incubation was performed at room temperature for 30 minutes, 1 hour, 2 hours, 3 hours, and 21 hours, respectively. The ADP concentrations and fluorescence values were fitted using GraphPad Prism 8 before the calculation of EC₅₀. The H8-9 dose curve is shown in FIG. 7. Furthermore, the readings for each concentration series at 30 minutes, 1 hour, 2 hour, and 3 hours almost overlap with the dose curve, indicating high stability in fluorescence intensity.

The fluorescence signal of the present disclosure is stable. At an H8-9 concentration of 10 nM, the reading after 21 hours of incubation was 1.8×10⁶, still greater than 50% of the readings at 30 minutes of incubation (3×10⁶), and the EC₅₀ remained unchanged.

The EC₅₀ of H8-9 against PKA was detected using the ADP-Glo kit purchased from Promega. The results are shown in Table 5. The results of the present disclosure are consistent with those detected by the ADP-Glo kit.

TABLE 5
Comparison of H8-9 EC₅₀ values detected by different protocols

H8-9 EC₅₀ (nM)

| | This disclosure | Promega ADP-Glo |
|---|---|---|
| ATP 10 μM | 94 | 65 |
| ATP 100 μM | 383 | 567 |

### 2. EC₅₀ of PKI Against PKA.

PKI was serially diluted by 3-fold using the universal kinase reaction buffer, to a maximum concentration of 1 μM and a minimum concentration of 0.03 nM.

ATP concentration of 10 μM:

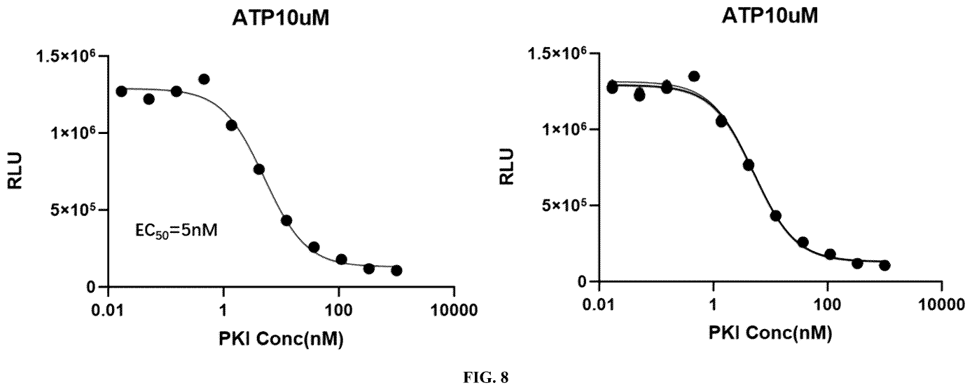

25 μL of the universal kinase reaction buffer including 80 U PKA and a varied concentration of PKI was added to each well of a 96-well white opaque assay plate, and 10 μM ATP was added. The reaction was performed at room temperature (22° C.-25° C.) for 10 minutes. After the reaction was finished, 25 μL of the reagent for ATP removal was added. After incubation at room temperature (22° C.-25° C.) for 40 minutes, 50 μL of the reagent for ADP detection was added and mixed uniformly, and the fluorescence values were read after incubation was performed at room temperature for 30 minutes, 1 hour, 2 hours, 3 hours, and 21 hours, respectively. The ADP concentrations and fluorescence values were fitted using GraphPad Prism 8 before the calculation of EC₅₀. The PKI dose curve is shown in FIG. 8. Furthermore, the readings for each concentration series at 30 minutes, 1 hour, 2 hour, and 3 hours almost overlap with the dose curve, indicating high stability in fluorescence intensity.

The fluorescence signal of the present disclosure is stable. At an PKI concentration of 1 nM, the reading after 21 hours of incubation was 7.4×10⁵, still greater than 50% of the readings at 30 minutes of incubation (1.4×10⁶), and the EC₅₀ remained unchanged.

ATP concentration of 100 μM:

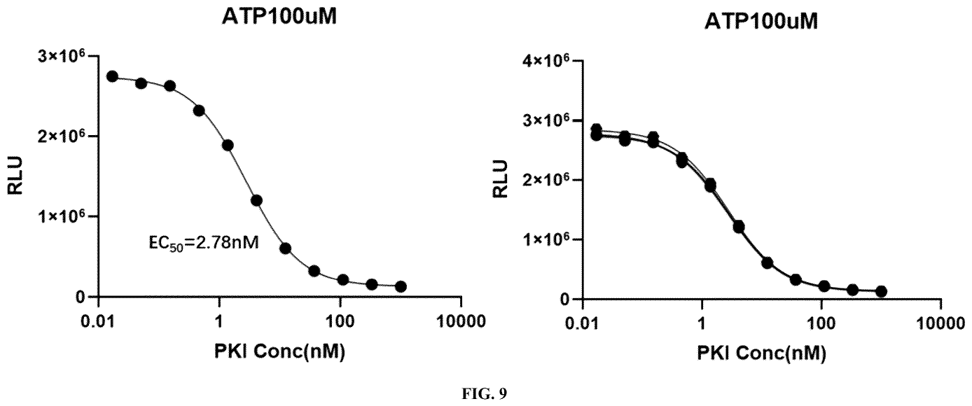

25 μL of the universal kinase reaction buffer including 80 U PKA and a varied concentration of PKI was added to each well of a 96-well white opaque assay plate, and 100 μM ATP was added. The reaction was performed at room temperature (22° C.-25° C.) for 30 minutes. After the reaction was finished, 25 μL of the reagent for ATP removal was added. After incubation at room temperature (22° C.-25° C.) for 40 minutes, 50 μL of the reagent for ADP detection was added and mixed uniformly, and the fluorescence values were read after incubation was performed at room temperature for 30 minutes, 1 hour, 2 hours, 3 hours, and 21 hours, respectively. The ADP concentrations and fluorescence values were fitted using GraphPad Prism 8 before the calculation of EC₅₀. The PKI dose curve is shown in FIG. 9. Furthermore, the readings for each concentration series at 30 minutes, 1 hour, 2 hour, and 3 hours almost overlap with the dose curve, indicating high stability in fluorescence intensity.

The fluorescence signal of the present disclosure is stable. At an PKI concentration of 0.03 nM, the reading after 21 hours of incubation was 1.6×10⁶, still greater than 50% of the readings at 30 minutes of incubation (2.7×10⁶), and the EC₅₀ remained unchanged.

The EC₅₀ of PKI against PKA was detected using the ADP-Glo kit purchased from Promega. The results are shown in Table 6. The results of the present disclosure are consistent with those detected by the ADP-Glo kit.

TABLE 6
Comparison of PKI EC₅₀ values detected by different protocols

PKI EC₅₀ (nM)

| | This disclosure | Promega ADP-Glo |
|---|---|---|
| ATP 10 μM | 5 | 10 |
| ATP 100 μM | 2.78 | 4 |

A phosphotransferase (or ATP hydrolase) can consume the substrate ATP to generate the product ADP, and the activity of a phosphotransferase (or ATP hydrolase) is positively correlated with the yield of ADP. The activity of a phosphotransferase or an ATP hydrolase can be determined by measuring the yield of ADP after the phosphotransferase (or ATP hydrolase) reaction. If a modulator of a phosphotransferase (or an ATP hydrolase) is present in the reaction when ATP is consumed using a phosphotransferase (or an ATP hydrolases), the effect of the modulator on the phosphotransferase (or an ATP hydrolase) can be determined by measuring the yield of ADP after the phosphotransferase (or ATP hydrolase) reaction.

Compounds H8-9 and PKI are specific inhibitors of protein kinase A (PKA), where H8-9 is a competitive inhibitor of ATP. As the ATP concentration increases, the inhibitory effect of H8-9 reduces, i.e., the EC₅₀ value increases significantly. PKI is a non-competitive inhibitor of ATP, and its EC₅₀ is independent of the ATP concentration.

In the PKA kinase reaction, PKA consumes the substrate ATP to generate the product ADP, and the kinase activity is positively correlated with the yield of ADP. By measuring the yield of ADP after the PKA kinase reaction, the activity of PKA and the inhibitory effects of H8-9 and PKI on the kinase can be determined. The present disclosure removes the remaining ATP using ATPS and PPA after the PKA kinase reaction, and then converts the product ADP into ATP via a pyruvate kinase. The yield of ADP can be determined by detecting the amount of ATP converted.

The measured EC₅₀ values of H8-9 and PKI also indicate that by comparing the EC₅₀ values under different ATP concentrations, the competitive and non-competitive inhibitors of ATP are distinguished in the present disclosure.

### Example 5

The EC₅₀ values of the broad-spectrum kinase inhibitor Staurosporine (STSP) against PKA were measured.

The detection was performed using the reagent of Example 1.

Reagent for ATP removal: 50 mM Tris-HCl buffer (pH 7.4)+10 mM MgCl₂+20 mM Na₂MoO₄+1 μg/mL ATPS+10 U/mL PPA, where the concentration of each component is the final concentration in the reagent.

Reagent for ADP detection: 50 mM Tris-HCl buffer (pH 7.4)+20 mM MgSO₄+50 mM KCl+2 mM EDTA+2 mM EGTA+0.1 mM D-luciferin+0.1 mM PEP-K+50 μg/mL luciferase+20 U/mL a pyruvate kinase, where the concentration of each component is the final concentration in the reagent.

Universal kinase reaction buffer: 50 mM Tris buffer (pH 7.4)+20 mM MgCl₂+0.1 mg/mL BSA, where the concentration of each component is the final concentration in the buffer.

The bioluminescence detection instrument was an EnVision (Perkin Elmer).

STSP was serially diluted by 3-fold using the universal kinase reaction buffer, to a maximum concentration of 1 μM and a minimum concentration of 0.03 nM.

ATP concentration of 1 μM:

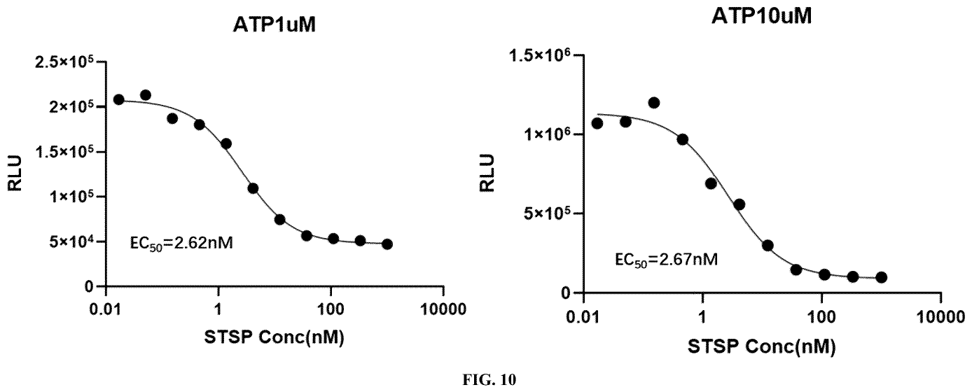

25 μL of the universal kinase reaction buffer including 80 U PKA and a varied concentration of STSP was added to each well of a 96-well white opaque assay plate, and 1 μM ATP was added. The reaction was performed at room temperature (22° C.-25° C.) for 30 minutes. After the reaction was finished, 25 μL of the reagent for ATP removal was added. After incubation at room temperature (22° C.-25° C.) for 40 minutes, 50 μL of the reagent for ADP detection was added and mixed uniformly, and the fluorescence values were read after incubation was performed at room temperature for 30 minutes and 19 hours, respectively. The ADP concentrations and fluorescence values were fitted using GraphPad Prism 8 before the calculation of EC₅₀. The STSP dose curve is shown in the left panel of FIG. 10. The EC₅₀ of STSP against PKA was 2.62 nM at an ATP concentration of 1 μM. This result was consistent with the EC₅₀=3.51 nM as measured using Promega's ADP-Glo reagent.

The fluorescence signal of the present disclosure is stable. At an STSP concentration of 0.03 nM, the reading after 19 hours of incubation was 1.3×10⁵, still greater than 50% of the readings at 30 minutes of incubation (2.2×10⁶), and the EC₅₀ remained unchanged.

ATP concentration of 10 μM:

25 μL of the universal kinase reaction buffer including 80 U PKA and a varied concentration of STSP was added to each well of a 96-well white opaque assay plate, and 100 μM ATP was added. The reaction was performed at room temperature (22° C.-25° C.) for 30 minutes. After the reaction was finished, 25 μL of the reagent for ATP removal was added. After incubation at room temperature (22° C.-25° C.) for 40 minutes, 50 μL of the reagent for ADP detection was added and mixed uniformly, and the fluorescence values were read after incubation was performed at room temperature for 30 minutes and 19 hours, respectively. The ADP concentrations and fluorescence values were fitted using GraphPad Prism 8 before the calculation of EC₅₀. The STSP dose curve is shown in the right panel of FIG. 10. The EC₅₀ of STSP against PKA was 2.67 nM at an ATP concentration of 10 μM. This result was consistent with the EC₅₀=3.63 nM as measured using Promega's ADP-Glo reagent.

The fluorescence signal of the present disclosure is stable. At an STSP concentration of 0.03 nM, the reading after 19 hours of incubation was 6.7×10⁵, still greater than 50% of the readings at 30 minutes of incubation (1.2×10⁶), and the EC₅₀ remained unchanged.

In summary, embodiments of the present disclosure can be effectively used for the detection of the phosphotransferase or ATP hydrolase activity, and has the advantages of high sensitivity, excellent linearity, and stable fluorescence signal.

The above example is merely preferred embodiments of the present disclosure. It should be noted that a person of ordinary skill in the art may make some improvements and modifications without departing from the principle of the present disclosure, and these improvements and modifications should also be regarded as falling within the protection scope of the present disclosure.

What is claimed is:

1. A reagent for detecting adenosine diphosphate (ADP), comprising an adenosine triphosphate (ATP) sulfurylase, a pyrophosphatase, an enzyme inhibitor, an enzyme that catalyzes the conversion of ADP to ATP, a substrate for the enzyme, a bioluminescent enzyme, and a substrate for the bioluminescent enzyme, wherein the enzyme inhibitor inhibits the activity of ATP sulfurylase and pyrophosphatase.

2. The reagent according to claim 1, wherein the nucleotide sequence of a gene encoding the ATP sulfurylase is set forth in SEQ ID NO: 1.

3. The reagent according to claim 1, wherein the enzyme that catalyzes the conversion of ADP to ATP is any one of a myokinase, a creatine kinase, or a pyruvate kinase, and the substrate for the enzyme is a phosphate donor that is capable being used by the enzyme.

4. The reagent according to claim 1, wherein the enzyme inhibitor comprises ethylenediaminetetraacetic acid (EDTA) and ethylene glycol tetraacetic acid (EGTA).

5. A kit for detecting ADP, comprising the reagent according to claim 1 and an ADP/ATP mixture standard.

6. A method for detecting ADP, wherein detection is performed using the reagent according to claim 1, comprising steps as follows:
removing ATP from a sample using the ATP sulfurylase and the pyrophosphatase;
converting ADP in the sample into ATP using the enzyme that catalyzes the conversion of ADP to ATP; and
detecting ATP in the sample using a bioluminescence reaction.

7. The method according to claim 6, wherein the nucleotide sequence of a gene encoding the ATP sulfurylase is set forth in SEQ ID NO: 1.

8. The method according to claim 6, wherein the enzyme that catalyzes the conversion of ADP to ATP is any one of a myokinase, a creatine kinase, or a pyruvate kinase, and the substrate for the enzyme is a phosphate donor that is capable being used by the enzyme.

9. The method according to claim 6, wherein the enzyme inhibitor comprises ethylenediaminetetraacetic acid (EDTA) and ethylene glycol tetraacetic acid (EGTA).

10. The method according to claim 6, wherein after removal of ATP from the sample, the activity of ATP sulfurylase and pyrophosphatase is inhibited using the enzyme inhibitor.

11. A method for detecting ADP, wherein detection is performed using the kit according to claim 5, comprising steps as follows:
removing ATP from a sample using the ATP sulfurylase and the pyrophosphatase;
converting ADP in the sample into ATP using the enzyme that catalyzes the conversion of ADP to ATP; and
detecting ATP in the sample using a bioluminescence reaction.

12. A method for detecting enzyme activity, comprising using the reagent according to claim 1, wherein the enzyme comprises a phosphotransferase and an ATP hydrolase.

13. The method according to claim 12, wherein the nucleotide sequence of a gene encoding the ATP sulfurylase is set forth in SEQ ID NO: 1.

14. The method according to claim 12, wherein the enzyme that catalyzes the conversion of ADP to ATP is any one of a myokinase, a creatine kinase, or a pyruvate kinase, and the substrate for the enzyme is a phosphate donor that is capable being used by the enzyme.

15. The method according to claim 12, wherein the enzyme inhibitor comprises ethylenediaminetetraacetic acid (EDTA) and ethylene glycol tetraacetic acid (EGTA).

16. A method for detecting and/or screening for a modulator of enzyme activity, comprising using the reagent according to claim 1, wherein the enzyme includes a phosphotransferase and an ATP hydrolase.

17. The method according to claim 16, wherein the nucleotide sequence of a gene encoding the ATP sulfurylase is set forth in SEQ ID NO: 1.

18. The method according to claim 16, wherein the enzyme that catalyzes the conversion of ADP to ATP is any one of a myokinase, a creatine kinase, or a pyruvate kinase, and the substrate for the enzyme is a phosphate donor that is capable being used by the enzyme.

19. The method according to claim 16, wherein the enzyme inhibitor comprises ethylenediaminetetraacetic acid (EDTA) and ethylene glycol tetraacetic acid (EGTA).

20. The method according to claim 16, wherein the modulator includes an enzyme inhibitor and an enzyme activator.
